(19) United States
(12) Patent Application Publication
(10) Pub. No.: US 2019/0231742 A1
(43) Pub. Date: Aug. 1, 2019

(54) NUTRITIONAL AGENTS USED TO PREVENT AND TREAT HYPERSENSITIVITY AND OTHER ALLERGIC-TYPE DISORDERS

(71) Applicant: Theodore B. VANITALLIE, Old Lyme, CT (US)

(72) Inventor: Theodore B. VANITALLIE, Old Lyme, CT (US)

(21) Appl. No.: 15/952,157

(22) Filed: Apr. 12, 2018

Related U.S. Application Data
(60) Provisional application No. 62/487,998, filed on Apr. 20, 2017.

(51) Int. Cl. A61K 31/381; A61P 37/08; A61K 9/00; A61K 31/12; A61K 31/22; A61K 31/045

(52) U.S. Cl. CPC A61K 31/381; A61P 37/08; A61K 31/045; A61K 31/12; A61K 31/22; A61K 9/0053

(57) ABSTRACT

A nutritional agent that orally delivers a therapeutically effective amount of a ketone body selected from the group consisting of D-β-hydroxybutyrate (D-βOHB), acetoacetate (AcAc), acetone and any of their isoforms, in a carrier vehicle, to prevent and treat hypersensitivity and allergic disorders.

This application claims the benefit of U.S. provisional application Ser. No. 62/487,998 filed Apr. 20, 2017 which is incorporated herein in its entirety by reference.

FIELD OF THE INVENTION

The present invention relates to nutritional agents used to stabilize mast cells and other granulocytes for the purpose of managing hypersensitivity reactions and other allergic-type disorders. More specifically, the invention provides dietary supplements that act to deliver the ketone bodies, D-⊖-hydroxybutyrate (D-⊖OHB) and/or acetoacetate (AcAc) to the human circulation via the gastrointestinal tract. This approach can be uniquely helpful in the prevention and nutritional management of an array of prevalent allergy-hypersensitivity states including hay fever, asthma, allergic eczema, atopic dermatitis, medication allergy and anaphylaxis.

### BACKGROUND OF THE INVENTION

In a 2014, a group of Japanese scientists (Nakamura et al.) published an article entitled, “Fasting mitigates immediate hypersensitivity: a pivotal role of endogenous D-beta-hydroxybutyrate (D-βOHB)” [Nakamura S, Hisamura R, Shimoda S, Shibuva I, Tsubota K. Fasting mitigates immediate hypersensitivity: a pivotal role of endogenous D-beta-hydroxybutyrate. Nutr Metab (Lond).2014;11:40-53.] The article presented evidence, using mice and rats, that the rise in D-βOHB which results from fasting plays an important role in the causation of this mitigation effect. Nakamura et al. suggested that the reduction in hypersensitivity results from D-βOHB's stabilization of the body's mast cells. Such stabilization prevents or attenuates the mast cell degranulation and the release from mast cells of histamine and other bioactive agents ordinarily induced in allergic individuals upon exposure to any of a host of allergenic agents (e.g. pollen, dander, bee venom, penicillin, etc.). By causing such stabilization of granulocytes (including mast cells), elevation of circulating blood levels of D-βOHB—at sufficient concentrations—prevents or mitigates hypersensitivity to allergens of various kinds. Fasting (which elevates blood levels of ketone bodies) also is known to enhance immunological defenses by attenuating the inflammation associated with allergic responses.

The in vitro and animal studies reported by Nakamura et al their article are comprehensive and convincing. Summaries of these studies are as follows:

Nasal allergy: In the toluene 2,4-diisocyanate (TDI)-sensitized nasal allergy rat model, fasting treatment for 24 hours attenuated the nasal hypersensitivity reaction (nasal rubbing, sneezing, and nasal [mucus] secretion, and elevated serum IgE concentration) that occurred in the nonfasted control animals. The histological observation disclosing that the nasal-maxilloturbinate mast cells were non-degranulated following 24-hours of fasting with TDI supported the results of suppressed nasal hypersensitivity reaction by fasting.

Systemic anaphylaxis: To examine the effect of food restriction on systemic anaphylaxis, Nakamura et al. evaluated antigen or Compound 48/80, an anaphylaxis inducer, on the resulting anaphylactic responses in immediate-type hypersensitivity by measuring reductions in rectal temperature. In the 24-hour fasting group, reductions in rectal temperature, an indicator of the anaphylactic reaction, were significantly smaller than those in the ad libitum group in both antigen- and Compound 48/80-induced anaphylaxis models.

Although it is possible to raise plasma D-βOHB and its sibling ketone body, acetoacetate [AcAc], by fasting or adherence to a ketogenic diet, these measures (with a few possible exceptions) are neither practical nor feasible as methods for use to prevent the hypersensitivity reaction from occurring in individuals with known or unknown allergies. It is possible to raise plasma D-βOHB levels to a mild degree (from ≤0.2 mM to ˜0.5-0.7 mM) by the oral administration of ˜20 g the medium-chain triglyceride (MCTG) tricaprylin (˜20 g). In striking contrast to this very modest rise caused by ingestion of MCTG are the 3 to 7 mM D-βOHB plasma concentrations achievable by prolonged fasting or adherence to a ketogenic diet.

In an article by Newport M T, Vanitallie T B (Applicant) et al [Alzheimer's Dement. 2015 Jan; 11(1): 99-103. Published online 2014 Oct 7. doi: 10.1016/j.jalz.2014.01.006] the response to 20 months of treatment with a novel ketone ester in a patient with advanced dementia of long standing is reported in detail.

The invention herein described embodies use of ketone esters, which are by far the best, most convenient, safest, fastest, and most potent modality for raising plasma D-βOHB concentrations to levels that would be expected to prevent, arrest, mitigate, attenuate, reverse hypersensitivity reactions in allergic individuals. Ketone esters are foods, not drugs, and can therefore be marketed as ‘medical foods’.

There is no reason to believe that the invention ketone ester treatment would interfere or conflict physiologically with the concurrent use of conventional therapies such as injections of epinephrine, ingestion of antihistamine medications, administration of glucocorticoids (prednisone), and the like.

With the development of ketone esters for oral administration, the invention makes it possible to maintain blood ketone levels within the range of 4-7 mM by taking a serving of ˜35-50 g by mouth every 3 or 4 hours during the waking hours. Levels in this range should be high enough to prevent, arrest, reverse, attenuate, and treat a wide range of clinical problems that arise because of an allergic-hypersensitivity diathesis.

The invention ketone ester treatment does not interfere or conflict physiologically with the use of current conventional therapies—such as injections of epinephrine, ingestion of antihistamine medications, administration of glucocorticoids (prednisone)—used to alleviate and manage the signs and symptoms of attacks of allergy.

Current management approaches to allergy-hypersensitivity conditions such as hay fever and more serious conditions such as asthma, medication allergy and anaphylaxis are far from satisfactory. Medications such as antihistamine drugs and glucocorticoids have troublesome and sometimes serious symptomatic and physiological side effects. The only nutritional intervention recommended to date appears in the 2014 publication by Nakamura et al. cited earlier herein. The authors suggest that strict adherence to a ketogenic diet is a potentially effective non-pharmacologic approach to inhibiting allergic-hypersensitivity responses to environmental antigens. Unfortunately, ketogenic diets are difficult to prepare and adhere to and may—over the long term—have damaging side effects, including an increased risk of atherogenesis and cardiovascular disease. Fasting will raise blood ketone levels; however, fasting-induced hyperketonemia only occurs when liver glycogen stores become exhausted—a process which takes several days. The many disadvantages of the use of fasting to generate therapeutic hyperketonemia are obvious and well known.

As R. L. Veech has pointed out in the article, Ketone effects on metabolism and transcription. J Lipid Res 2014;55:2004-8, “Ketosis induced by feeding a ketogenic diet can have contradictory effects owing to the simultaneous elevation of both ketone bodies and free fatty acids. While the elevation of ketone bodies increases the energy of ATP hydrolysis by reducing the mitochondrial NAD couple and oxidizing the co-Q couple, thus increasing the redox span between site I and site II, the metabolism of fatty acids leads to a reduction of both mitochondrial NAD and also of mitochondrial Q, causing decreases in the ΔG of ATP hydrolysis. In contrast, feeding ketone body esters leads to pure ketosis unaccompanied by elevation of free fatty acids, producing a physiological state not previously seen in nature. The effects of pure ketosis on transcription and upon certain neurodegenerative diseases make this approach not only interesting but one of potential therapeutic value.”

In contrast to relying on consumption of a ketogenic diet to deal with the problems caused by allergy/hypersensitivity, the present invention is the first to call attention to the far greater effectiveness, rapidity of action and convenience of managing allergy/hypersensitivity by oral administration of a safe, tolerable, and readily digestible ketone ester delivered directly to the gastrointestinal tract as part of a molecule that may contain (for example) glycerol or 1,3-butanediol to render the D-βOHB, the active ingredient, nonirritating to the intestinal wall and nondamaging to the intestinal mucosa.

Thus, the invention consists of an entirely new nutritional application of certain novel diet supplements (such as ketone esters [KEs]), of which 2 have been awarded GRAS [‘generally recognized as safe’] status by the Food & Drug Administration [FDA]). The invention embodies the same special application of all other kinds of diet supplements whose physiological action is, or proves to be, similar or analogous in its action to that of D-β-hydroxybutyrate (D-βOHB) and acetoacetate (AcAc), including food products other than ketone esters that are ketogenic, or consist of, embody or otherwise carry such agents and deliver them safely to the gastrointestinal tract. Examples of such “other” products include butyric acid, which is reported to be ketogenic, ketone salts (e.g. the individual sodium, potassium, magnesium, or calcium salt of ketone bodies such as D-β-hydroxybutyrate [D-βOHB] and acetoacetate [AcAc], or various mixtures of such salts). All such formulations can serve this new use if they prove to be clinically safe and provided that their ultimate nutritional/biochemical action is to stabilize the granulocyte mechanism (i.e. mast cells, etc.) responsible for the release of histamine and/or other bioactive agents in response to exposure to various antigens/allergens that give rise to a range of allergic/hypersensitivity responses.

The invention involves use of physiologically benign carriers that act to deliver the nutrients D-β-hydroxybutyrate (D-βOHB) and/or acetoacetate (AcAc) to the human circulation via the gastrointestinal tract. This method for rapidly delivering ketone bodies to the bloodstream can be uniquely helpful in the nutritional management of an array of prevalent allergy-hypersensitivity states. Such states range from relatively mild but troublesome conditions such as hay fever to progressively more serious perturbations such as asthma, medication allergy and systemic anaphylaxis.

The new nutritional approach described by the invention herein and represented by the oral administration of the ketone body D-βOHB (in some instances together with AcAc) in a gastro-intestinally benign form, as a component of a suitable carrier/vehicle, represents a major advance in the nutritional management of a constellation of very common and often serious allergy/hypersensitivity conditions.

The magnitude and scope of the allergy-hypersensitivity problem are summarized by the data below provided by the American Academy of Allergy, Asthma, & Immunology [http://www.aaaai.org/about-aaaai/newsroom/allergy.statistics] presenting worldwide allergy statistics (2009 to 2012).

### Allergic Rhinitis

Roughly 7.8% of people 18 and over in the U.S. have hay fever.

In 2010, white children in the U.S. were more likely to have had hay fever (10%) than black children (7%).

Worldwide, allergic rhinitis affects between 10% and 30% of the population.

Worldwide, sensitization (IgE antibodies) to foreign proteins in the environment is present in up to 40% of the population.

In 2012, 7.5% or 17.6 million adults were diagnosed with hay fever in the past 12 months.

In 2012, 9.0% or 6.6 million children reported hay fever in the past 12 months.

In 2010, 11.1 million visits to physician offices resulted with a primary diagnosis of allergic rhinitis.

### Drug Allergy

Worldwide, adverse drug reactions may affect up to 10% of the world's population and affect up to 20% of all hospitalized patients.³

Worldwide, drugs may be responsible for up to 20% of fatalities due to anaphylaxis.

### Food Allergy

Findings from a 2009 to 2010 study of 38,480 children (infant to 18) indicated:
8% have a food allergy
Approximately 6% aged 0-2 years have a food allergy
About 9% aged 3-5 years have a food allergy
Nearly 8% aged 6-10 years have a food allergy
Approximately 8% aged 11-13 years have a food allergy
More than 8.5% aged 14-18 years have a food allergy

38.7% of food allergic children have a history of severe reactions

30.4% of food allergic children have multiple food allergies

Of food allergic children, peanut is the most prevalent allergen, followed by milk and then shellfish

In 2012, 5.6% or 4.1 million children reported food allergies in the past 12 months.

### General Allergy

Worldwide, the rise in prevalence of allergic diseases has continued in the industrialized world for more than 50 years.

Worldwide, sensitization rates to one or more common allergens among school children are currently approaching 40%-50%.

In 2012, 10.6% or 7.8 million children reported respiratory allergies in the past 12 months.

### Insect Allergy

Worldwide, in up to 50% of individuals who experience a fatal reaction there is no documented history of a previous systemic reaction.

### Sinusitis

Roughly 13% of people 18 and over in the U.S. have sinusitis.

### Skin Allergy

In 2010, black children in the U.S. were more likely to have had skin allergies (17%) than white (12%) or Asian (10%) children.

Worldwide, urticaria occurs with lifetime prevalence above 20%.

In 2012, 12.0% or 8.8 million children reported skin allergies in the past 12 months.

A general objective of the invention is to provide a novel, convenient, safe, and effective method of prevention, treatment and mitigation of the adverse effects resulting from allergic, hypersensitivity and anaphylactic disorders ranging from allergic rhinitis to asthma, food sensitivity, passive cutaneous anaphylaxis, to anaphylactic shock. These clinical conditions result from allergen-induced degranulation of certain of the body's granulocytes. Especially noteworthy among the granulocyes are the mast cells. In addition, other granulocytes, including basophils, eosinophils, and, possibly, specialized granule-containing polymorphonuclear (PMN) leucocytes, may also contribute to the hypersensitivity/allergic responses that occur in susceptible individuals upon exposure any of a host of allergens/antigens, and other sensitizing environmental or endogenous agents. Changes in the immunoglobulin, plasma IgE levels in the body, can serve as a marker of type 1 hypersensitivity which manifests in various allergic diseases such as asthma, types of sinusitis, allergic rhinitis, food allergies, types of chronic urticaria and atopic dermatitis.

Another object of the invention is in the delivery (via oral administration and intestinal absorption) to the patient of D-(βOHB, and, to a lesser extent, its companion ketone body, acetoacetate (AcAc). The relationship of these two sibling molecules is described on pages 666-7 of Lehninger's Principles of Biochemistry, 5^{th} Edition, by David L. Nelson and Michael M. Cox, and published by W.H. Freeman & Co., New York, as follows:“The first step in the formation of acetoacetate, occurring in the liver, is the enzymatic condensation of two molecules of acetyl-CoA to form β-hydroxy-β-methyl glutaryl-CoA (HMG-CoA), which is cleaved to free acetoacetate and acetyl-CoA. The acetoacetate is reversibly reduced by D-β-hydroxybutyrate dehydrogenase, a mitochondrial enzyme, to D-β-hydroxybutyrate. This enzyme is specific for the D-stereoisomer, it does not act on L-β-hydroxyacyl-CoAs.”

Ketones are appropriately regarded as nutrients in the sense that the glucose which, like ketone bodies, circulates in the blood and which provides energy to most of the body's cells and organs, is considered to be a bona fide nutrient, not a drug. This “nutritional approach” to the management of allergic and allergy/hypersensitivity disorders has the advantage of being physiologically based, with an excellent safety profile. The invention treatment also has the advantage of lacking the undesirable side effects that can occur with current anti-allergy, anti-histamine treatments, such as dry mouth, drowsiness, dizziness, nausea and vomiting, restlessness and moodiness, trouble urinating, blurred vision and confusion. The treatment also avoids the risks and adverse side effects associated with the administration of epinephrine and other sympathomimetic drugs and use of potent glucocorticoids like prednisone. At the same time, there seems to be no contraindication to administering a D-βOHB carrier such as a ketone ester along with the currently used treatment modalities.

### SUMMARY OF THE INVENTION

In the present invention, these purposes, as well as others which will be apparent, are achieved generally by providing a nutritional agent that, taken by mouth, carries a therapeutically effective amount of a ketone body to the intestinal tract attached to a carrier vehicle designed to obviate an irritative or other kind of damage to the intestinal mucosal lining, and at the same time prevent, mitigate, and treat hypersensitivity and allergic type disorders.

The ketone body used in the invention is selected from the ketone family, consisting of D-β-hydroxybutyrate (D-βOHB), acetoacetate (AcAc), acetone and any of their isoforms. The isoforms used in the invention are clinically safe for human consumption.

The nutritional agent according to the invention is used to prevent, mitigate, and treat disorders including hay fever, asthma, allergic eczema, atopic dermatitis, medication allergy and anaphylaxis.

In general, the carrier vehicle for the ketone body is gastro-intestinally benign. The carrier vehicle is preferably the ester moiety of a ketone ester, known to be safe for human consumption, or (less desirably) a member of the alkali metal group moieties (Na⁺, K⁺, Ca²⁺, etc.) of ketone salts currently in use or under investigation. By creation of a carrier vehicle that removes the harmful acidity from such moieties, the ketone body moiety of the carrier vehicle is made safer for human consumption.

The ketone esters used in the invention are preferably selected from the group consisting of 1, 3 butanediol monoester of D-βOHB, known as ketone monoester (KME), and glycerol tri-ester of betahydroxbutyrate (see Hashim and Vanitallie, J Lipid Res, 2014;55:1818-26.)

While these are preferred esters that have been and are being studied, the invention also embodies all future ketone esters, and other ketone salts that may be developed in the future, which are capable of being used for prevention, treatment and attenuation of sensitivity-allergy conditions, and which have been approved by the US FDA.

The invention also embodies molecules that function like ketone esters and ketone salts that can be given safely by mouth and which are capable of delivering ketone bodies into the intestinal lumen or wall without irritating or damaging intestinal mucosal lining.

Once ingested by the user, the carrier vehicle is digested and metabolized so as to free the ketone body component for absorption from the intestinal lumen and prompt transfer into the circulatory system. After digestion of the ketone esters or salts has taken place in the small intestine, the newly released ketone bodies can be safely absorbed into the venous system that drains water-soluble nutrients and other metabolites from the intestine. The ketone bodies are then transported to the systemic circulation from which they can access the granulocytes that participate in the allergic/sensitivity responses to allergens and antigens. The ketone bodies also access and cross the Blood Brain Barrier (BBB) for entry into the brain. The ketone salts used in the invention are selected from the group consisting of ketone bodies combined with alkali metal group moieties such as Na⁺, K⁺, Ca²⁺, Mg²⁺.

The carrier vehicle may further include at least one of the components selected from the group consisting of glycerol, phospholipids, triglycerides, partial glycerides, lipoproteins, medium-chain fatty acids and lauric acid. Other carrier vehicles for delivery of ketone bodies to the intestine for digestion and absorption may be produced in the future. If their safety and efficacy are properly established, such products would be covered by the claims detailed in this patent.

However, these patent provisions are also applicable to and cover timed- or delayed-release versions of ketone esters designed for treatment, prevention, amelioration, attenuation and other beneficial effects on sensitivity-allergy disorders such as those described in this document.

The ketone bodies themselves, even when combined with a taste-neutral ester moiety like glycerol, generally have a disagreeable taste that needs to be masked in order to make the ketone ester more palatable. For example, the nutritional agent can be incorporated into a tasty liquid beverage or food product. Various flavors, including citrus, have been found useful for improving the taste. Other approaches also may be effective.

Once in the circulatory system the ketone body can access and stabilize and inhibit or attenuate the activation of mast cells, basophils and eosinophils, which upon exposure to one or more allergens/antigens, may respond by extruding their granules. Essentially, a major effect of the ketone body is to block or inhibit granular extrusion.

The ketone body further inhibits the degranulation and release from mast cells, basophils, eosinophils (and any other granulocytes that respond to allergens and allergen-like agents) of granules with their content of such bioactive agents as histamine, prostaglandins, and other bioactive agents. Therapeutically effective amounts of ketone bodies would be those that are sufficient to generate ketone body blood levels in the range of approximately 0.5 mM to 7 mM.

The invention also provides a method to prevent and treat allergy hypersensitivity disorders comprising the steps of administering a nutritional agent that orally delivers a therapeutically effective amount of a ketone body in a carrier vehicle. Once in the bloodstream, the ketone bodies (D-βOHB and/or AcAc) in sufficient concentrations after absorption from the intestine, act to stabilize mast cells, basophils and eosinophils and thereby inhibit the release of bioactive agents from said mast cells, basophils and eosinophils contained within specific granules that would ordinarily be extruded when the granulocyte in question responds to an encounter with one or more invading allergens and antigens that are capable of stimulating degranulation.

The method provides blood plasma levels of said ketone body in the range of 0.5 mM to 7 mM. The nutritional agent is taken by a user every 3 to 4 hours to keep blood plasma levels of said ketone body in the desired part of the 0.5 mM to 7 mM range.

The bioactive agent in the invention is selected from the group consisting of histamine, prostaglandins and other bioactive agents that are secreted in response to allergens and allergen-like agents.

Other objects, features and advantages of the present invention will be apparent when the detailed description of the preferred embodiments of the invention are considered with reference to the drawings, which should be construed in an illustrative and not a limiting sense.

### BRIEF DESCRIPTION OF THE DRAWINGS

FIG. 1 shows the relative abundance of mediators, including histamine, leukotrienes, heparin, and many proteases released from IgE-primed mast cells, which are collectively responsible for the characteristic symptoms of allergy [figure taken from the 2012 paper by Amin K. The role of mast cells in allergic inflammation. Respiratory Medicine 2012;106:9-14];

FIG. 2 shows the induction and effector mechanism (principally cytokines) in type 1 hypersensitivity that give rise to allergic inflammation [taken from the 2012 paper by Amin K. The role of mast cells in allergic inflammation. Respiratory Medicine 2012;106:9-14];

FIG. 3 illustrates graphs of D-βOHB and AcAc concentrations taken from a paper published by Kieran Clarke PhD, a professor at the University of Oxford and Vanitallie (Applicant herein). The graphs show that as the amount of ketone monoester (KME) taken by mouth (in mg/kg body weight) increases in quantity the rise in the blood level of the two ketone bodies (D-βOHB and AcAc) increases proportionately; and

FIG. 4 is a graphic illustration of the time course of changes in blood D-βOHB concentration after an Alzheimer's disease patient ingested increasing quantities of D-βOHB [ketone] monoester [KME] on successive days. Note that the peak levels achieved seem to occur about 1 hour after KME ingestion. During the three hours after ingestion of the largest quantity (50 g), D-βOHB blood concentrations remained between 4.8 and 7 mM [taken from the 2014 paper by Newport, MT et al. A new way to produce hyperketonemia: Use of ketone ester in a case of Alzheimer's disease. Alzheimer's & Dementia 2014;1-5].

### DETAILED DESCRIPTION OF THE INVENTION

In accordance with the present invention, nutritional agents, including medical foods, are provided for use, now and/or in the future, in the nutritional management of allergy-hypersensitivity conditions that involve the release of histamine and other bioactive agents from the body's mast cells. Specifically, such management can be accomplished by the oral administration (including by enteric tube feeding when necessary) of suitable carrier vehicles that incorporate ketone bodies such as D-β-hydroxybutyrate (D-βOHB), acetoacetate (AcAc), acetone and/or any of their physiologically appropriate isoforms. The isoforms used in the invention are those that are clinically safe for human consumption.

Mast cells, basophils, and eosinophils play key roles in many kinds of inflammatory reactions, particularly those that cause clinically identifiable allergic responses in susceptible individuals. To deal with such problems, the invention provides a nutritional agent that delivers a therapeutically effective amount of a ketone body that can inhibit or attenuate the allergy/sensitivity responses to allergenic triggers in the body. Each of these granule-containing cells contains its own inventory of bioactive agents that act in the body to produce a variety of allergic responses. These inventories and the responses they generate are discussed below. Basophils, for example, contain and secrete by degranulation, the anticoagulant, heparin and the vasodilator, histamine (also secreted by mast cells). The pathological roles of mast cells, basophils, and eosinophils in the allergy scenario are either directly or indirectly linked to the presence of allergen-specific IgE in allergic individuals.

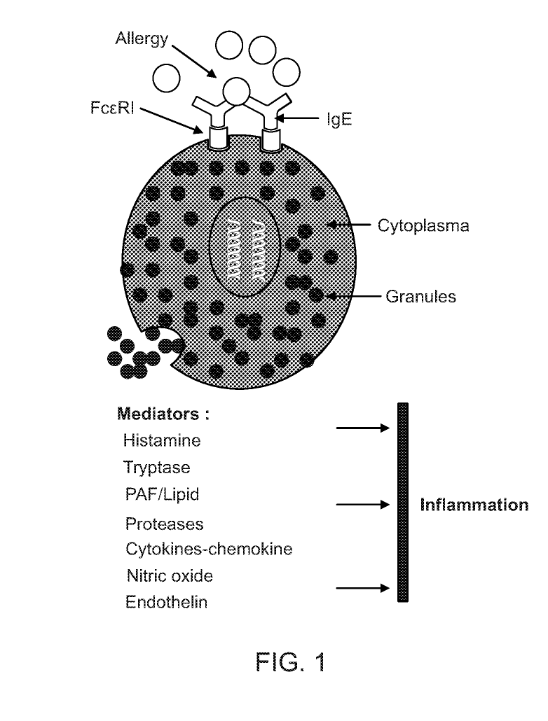

FIG. 1 shows the relative abundance of mediators, including histamine, leukotrienes, heparin, and many proteases released from IgE-primed mast cells, which are collectively responsible for the characteristic symptoms of allergy [figure taken from the 2012 paper by Amin K. The role of mast cells in allergic inflammation. Respiratory Medicine 2012;106:9-14].

MAST CELL. A mast cell (also known as a mastocyte or a labrocyte) is a type of white blood cell. Specifically, it is a type of granulocyte derived from the myeloid stem cell that is a part of the immune and neuroimmune systems and contains many granules rich in histamine and heparin. Mast cells provide a role in allergy and anaphylaxis as well as a protective role, being intimately involved in wound healing, angiogenesis, immune tolerance, defense against pathogens, and blood-brain barrier function.

The mast cell is very similar in both appearance and function to the basophil, another type of white blood cell. Although mast cells were once thought to be tissue resident basophils, it has been shown that the two cells develop from different hematopoietic lineages and thus cannot be the same cells

Mast cells are very similar to basophil granulocytes (a class of white blood cells) in blood. Both are granulated cells that contain histamine and heparin, an anticoagulant. The Fc (fragment crystallization) region of immunoglobulin E (IgE) becomes bound to mast cells and basophils and when IgE's paratopes bind to an antigen, it causes the cells to release histamine and other inflammatory mediators. These similarities have led many to speculate that mast cells are basophils that have “homed in” on tissues. Furthermore, they share a common precursor in bone marrow expressing the CD34 molecule. Basophils leave the bone marrow already mature, whereas the mast cell circulates in an immature form, only maturing once in a tissue site. The site an immature mast cell settles in probably determines its precise characteristics.

BASOPHILS. Basophils are a type of white blood cells. Basophils are the least common of the granulocytes, representing about 0.5 to 1% of circulating white blood cells. However, they are the largest type of granulocyte. They are responsible for inflammatory reactions during immune response, as well as in the formation of acute and chronic allergic diseases, including anaphylaxis, asthma, atopic dermatitis and hay fever. They can perform phagocytosis (cell eating), and secrete histamine and serotonin agents that induce/promote inflammation, and heparin that prevents blood clotting.

EOSINOPHILS. Eosinophils sometimes called eosinophiles or, less commonly, acidophils, are a variety of white blood cells and one of the immune system components responsible for combating multicellular parasites and certain infections in vertebrates. Along with mast cells and basophils, they control mechanisms associated with allergy and asthma. They are granulocytes that develop during hematopoiesis in the bone marrow before migrating into blood, after which they are terminally differentiated and do not multiply. These cells are eosinophilic or “acid-loving” due to their large acidophilic cytoplasmic granules.

In normal individuals, eosinophils make up about 1-3% of white blood cells and are about 12-17 μm in size with bilobed nuclei. While they are released into the bloodstream as neutrophils are, eosinophils reside in tissue. They are found in the medulla and the junction between the cortex and medulla of the thymus, and, in the lower gastrointestinal tract, ovary, uterus, spleen, and lymph nodes, but not in the lung, skin, esophagus, and a few other organs. The presence of eosinophils in these latter organs is associated with disease. For instance, patients with eosinophilic asthma have high levels of eosinophils that lead to inflammation and tissue damage, making it more difficult for patients to breathe.

Allergies, also known as allergic diseases, are a number of conditions caused by hypersensitivity of the immune system to something in the environment that usually causes little or no problem in most people. These diseases include hay fever, food allergies, atopic dermatitis, allergic asthma and anaphylaxis. Symptoms may include red eyes, an itchy rash, sneezing, a runny nose, shortness of breath, or ‘hives’ (urticaria, an allergic skin response) characterized by swelling and itching of the skin.

Common allergens include pollen and certain foods. Metals and other substances may also cause allergy problems. Food, insect stings, and medications are common causes of severe reactions. Their development is due to both genetic and environmental factors. The underlying mechanism involves immunoglobulin E antibodies (IgE), part of the body's immune system, binding to an allergen and then to a receptor on mast cells or basophils which triggers the release of inflammatory chemicals such as histamine. Diagnosis is typically based on a person's medical history and testing of the skin or blood may be useful in certain cases. Positive tests, however, may not mean there is a significant allergy to the substance in question.

Early exposure to potential allergens may be protective. Treatments for allergies include avoiding known allergens and the use of medications such as steroids and antihistamines. In severe reactions injectable adrenaline (epinephrine) is recommended. Allergen immunotherapy, which gradually exposes people to larger and larger amounts of allergen, is useful for some types of allergies such as hay fever and reactions to insect bites. Its use in food allergies is unclear. The invention nutritional agent provides an alternative to these known treatments.

Allergies are common. In the developed world, about 20% of people are affected by allergic rhinitis, about 6% of people have at least one food allergy, and about 20% have atopic dermatitis at some point in time. Depending on the country about 1-18% of people have asthma. Anaphylaxis occurs in between 0.05-2% of people. Rates of many allergic diseases appear to be increasing.”

### IgE and the Nomenclature of Allergic Disease

The understanding of the immunological mechanisms underlying allergic disease has led to a revised nomenclature, which relates clinical symptoms to the initiating immunological mechanism. The essence of this new nomenclature can be found in several language translations on the European Academy of Allergology and Clinical Immunology's website. Allergy is defined as “a hypersensitivity reaction mediated by immunological mechanisms” which can be antibody- or cell-mediated.

In the majority of cases the antibody typically responsible for an allergic reaction belongs to the IgE isotype and individuals may be referred to as suffering from an IgE-mediated allergic disease, eg, IgE-mediated asthma. Atopy is a personal or familial tendency to produce IgE antibodies in response to low doses of allergens, usually proteins, and, as a consequence, to develop typical symptoms of asthma, rhinoconjunctivitis or allergic skin disease. What is generally known as “atopic eczema/dermatitis” is not one, single disease but rather an aggregation of several diseases with certain clinical characteristics in common, and the term atopic eczema/dermatitis syndrome (AEDS) has been proposed. The subgroup related to allergic asthma and rhinoconjunctivitis, the IgE-associated subgroup of AEDS, can appropriately be called atopic dermatitis/atopic eczema.

### Sensitization

The immune response in allergy begins with sensitization. When, for example, house dust mites or pollen allergens are inhaled, antigen presenting cells in the epithelium lining of the airways of the lungs and nose, internalize, process and then express these allergens on their cell surface. The allergens are then presented to other cells involved in the immune response, particularly T-lymphocytes. Through a series of specific cell interactions B-lymphocytes are transformed into antibody secretory cells—plasma cells. In the allergic response, the plasma cell produces IgE-antibodies, which, like antibodies of other immunoglobulin isotypes, are capable of binding a specific allergen via its Fab ‘fragment antigen-binding’ portion. Different allergens stimulate the production of corresponding allergen-specific IgE antibodies. Once formed and released into the circulation, IgE binds, through its Fc ‘fragment crystallizable’ circulating’ portion, to high affinity receptors on mast cells, leaving its allergen specific receptor site available for future interaction with allergen. Other cells known to express high-affinity receptors for IgE include basophils, eosinophils, Langerhans cells and activated monocytes. Production of allergen-specific IgE-antibodies completes the immune response known as sensitization.

### Re-exposure to Allergen

Upon re-exposure, binding of the allergen to IgE orchestrates the immune system to initiate a more aggressive and rapid memory response. Cross-linking of a sufficient number of mast cell/basophil-bound IgE antibodies by allergen initiates a process of intra-cellular signaling, which leads to degranulation of cells, with the release of mediators of inflammation. Mast cells attempt to sustain a fixed number of unoccupied high-affinity IgE receptors on their cell surface. IgE antibodies bind to these receptor sites, waiting for their specific allergen to be encountered. To keep the number of unoccupied IgE receptor sites constant the mast cell regulates IgE receptor expression, probably in response to the levels of circulating IgE. Analogous responses are presumably expressed by basophils and eosinophils,

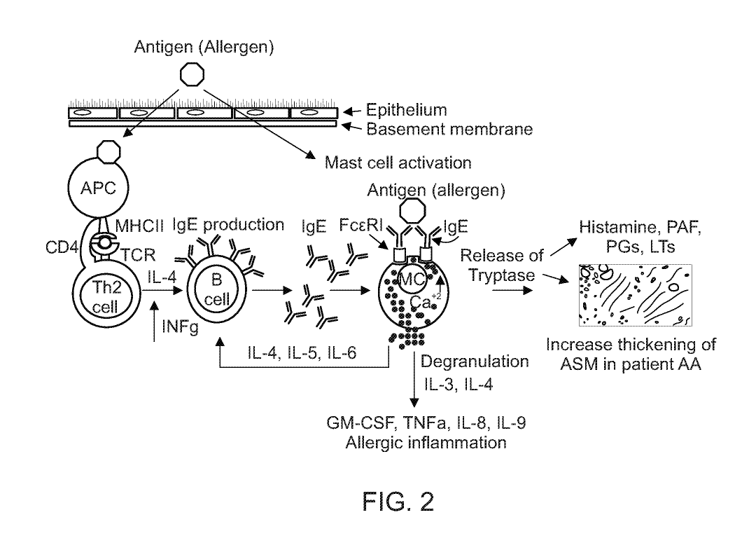

FIG. 2 shows the induction and effector mechanism (principally cytokines) in type 1 hypersensitivity that give rise to allergic inflammation [taken from the 2012 paper by Amin K. The role of mast cells in allergic inflammation. Respiratory Medicine 2012;106:9-14].

The ketone bodies used in the invention are preferably in a carrier vehicle. The preferred ketone bodies delivered to the body's blood circulation are D-βOHB or AcAc. These ketone bodies are both organic acids and, because they are acids, they are irritating to the intestine's mucosal lining and can actually cause physical damage to that lining. Combining the ketone body with glycerol or 1, 3-butanediol (as is done with the Hashim and Veech esters respectively) creates a ketone ester that is no longer “acidic” but, instead, “neutral”) [pH neutral] and therefore does not have the irritating “burning” effect of an acidic compound. That is why combining the D-βOHB (or AcAc) with a moiety like glycerol or 1, 3-butanediol, creates a carrier vehicle that does not irritate or damage the intestinal mucosa. If the ketone body was not delivered in this esterified form, the ketone body would not be tolerated by the patient. The ketone ester is digested in the small intestine releasing the ketone body which is absorbed through the intestinal wall and transported by capillaries and venous drainage blood vessels into the portal venous system and from there into the general blood circulation in the physiologically normal fashion.

Any molecules, nutrients, or agents that can serve as safe and effective carriers or vehicles for D-βOHB/AcAc/acetone loads that are taken by mouth and travel down the small intestine are embodied in the scope of this application. This proviso particularly applies to their roles as carriers of ketone bodies in the gastrointestinal tract. It is understood that when digestion frees the ketone body from its carrier, the ketone body is readily absorbed from the intestinal lumen into the portal vein, with subsequent rapid and efficient transfer into the systemic circulation.

The ketone bodies and the vehicles covered in this application include all ketone esters found to be safe for human administration now and in the future (for example, those that have been, or will be, given GRAS [generally recognized as safe] status by FDA).

The oral ketones and their carriers may also include such molecules as phospholipids, triglycerides, partial glycerides, lipoproteins and ketone body salts (of Na⁺, K⁺, Ca²⁺, Mg²⁺, etc. [as one molecule or as mixtures of single molecules]. Also included are clinically safe nutritive agents that are precursors of the ketone bodies such as medium-chain fatty acids which are obligatorily converted to ketone bodies following transport into the liver, butyric acid or lauric acid which stimulates the production of ketone bodies for transfer to brain neurons by central nervous system glial cells. Thus, ketone bodies may be incorporated into many different kinds of organic molecules that are safe to consume and may be released from such molecules upon their digestion in the intestinal tract or, on occasion, in the blood circulation.

As mentioned earlier, the carrier vehicle—glycerol—is a normal constituent of the human body. For example, when fat cells in depot fat release fatty acids for use as fuel they also release glycerol because fat is stored in the neutral form with one molecule of triglyceride being combined with up to three fatty acid molecules. The released glycerol travels in the circulation and can be converted to glycogen or glucose in the liver. When the ketone ester (e.g. the ketone body [an acid]) is chemically combined with a glycerol molecule (a pH neutral substance) and is digested in the intestinal tract, the glycerol that is released from the molecule is absorbed into the portal venous system (as is the ketone body) and metabolized in the liver—usually to glucose. The relevance is the fact that the glycerol moiety is a normal metabolite in the human body and is completely harmless, rendering the ketone body with which it is combined safe for ingestion by mouth.

Ketone bodies or ketone-body precursors are delivered to the gastrointestinal tract in various carriers that, when digested or otherwise metabolized, release ketone bodies (KBs) into the circulation. The ketone bodies act to stabilize mast cells and inhibit their degranulation in response to allergens and allergen-like agents. It will be recalled that such agents (like pollen, for example) generate various kinds of illness-producing allergic reactions mediated by the body's many mast cells. The ketone bodies, D-βOHB and AcAc (which ‘stabilize’ mast cells and inhibit their release of histamine and other agents that are part of the allergic response), are normal constituents of the body's physiology and circulation and, like the glucose that travels in the blood stream and nourishes the body's cells, they are normal constituents of the circulating blood. Like glucose, they are properly defined as foods rather than drugs. The proposed patent application does not envision the use of any ‘drug’ to control allergy, or, indeed, to have any drug in its purview.

The invention encompasses D-βOHB, acetoacetate (AcAc) and acetone (A), and related salts. Collectively, D-βOHB, acetoacetate and acetone are the molecules known as “ketone bodies”. All increase in the blood circulation during untreated diabetes mellitus, fasting, adherence to a ketogenic diet, and oral consumption of ketone esters and ketone salts, and other molecules that, after ingestion and during digestion in the small intestine release D-β-hydroxybutyrate (D-βOHB) [or acetoacetate (AcAc)] into the intestinal lumen. From there, the D-βOHB is absorbed into the portal vein which drains into the liver and from there is collected into the hepatic vein which drains into the inferior vena cava, and thence the systemic blood circulation. The liver is unable to utilize the D-βOHB and exports it unchanged.

Acetone is a breakdown product of AcAc and may have some ketone-like effects on aspects of the body's metabolism. It is a very minor part of the ketone body picture. D-βOHB is readily converted to AcAc and vice versa during the metabolic process by which these two ketone bodies are used as a source of energy (fuel), and may also act as metabolic signaling devices. The principal ketone body is D-βOHB. But D-βOHB is converted to AcAc before being metabolized in the Krebs tricarboxylic acid cycle. Because of their biochemical interconnections, the three ketone bodies are considered to be component parts of the same metabolic family.

When used as therapeutic agents, D-βOHB and AcAc appear to have the potential to provide the most direct approach to prevention, treatment, and mitigation of allergic-hypersensitivity responses (e.g. asthma, anaphylaxis, fibrosis, allergic rhinitis (hay fever), mast-cell caused irritable colon disease, and other allergen-generated illnesses. D-βOHB and AcAc basically act to stabilize mast cells and inhibit their release of histamine, cytokines, chemokines and similar agents when activated by exposure to suitable allergens.

Salts don't work better than ketone esters in the digestive tract and have the disadvantage of carrying a potentially unhealthy burden of sodium, potassium, etc. However, they are included herein because they have been used in patient care and are currently under study by investigators at the University of South Florida. Such agents could be used for the treatment, prevention, mitigation, etc. of allergy-hypersensitivity conditions. The salts also provide blood ketone elevating capability and are encompassed by the invention.

The carrier vehicles of the invention include the following examples.

Ketone Esters (Vehicles) that “Carry” BOHB

The 1,3 butanediol monoester of D-βOHB [structural formula shown in Table 1] has been approved for safety (GRAS) by the US FDA. It is being developed commercially at the University of Oxford in the United Kingdom for use as a source of efficient energy in sports activities, with evidence of enhancement of endurance and cognitive function found in rodents and in a smattering of Oxford athletes. This ester can be used in the invention to raise the whole blood level of D-βOHB to the 5-7 mM range, which is useful in treatment, prevention, and mitigation of allergic-type illnesses that would occur when mast cells become stimulated to release histamine, cytokines and other inflammatory agents in response to exposure to suitable allergy-inducing antigens.

TABLE 1

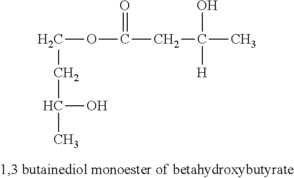

The D-βOHB molecule is the four (4) horizontally arranged carbons shown in the upper right. The butanediol moiety is the four (4) vertically arranged carbons shown on the far left. D-βOHB is quite acidic and too irritating to the gut to take by itself; hence, it is given orally in ester form, with the D-βOHB being esterified with the butanediol to form a “ketone ester.”

TABLE 2

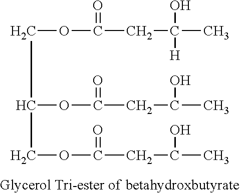 [SMILES: CC(O)CC(=O)OCC(COC(=O)CC(C)O)OC(=O)CC(C)O]

The glycerol tri-ester of D-βOHB, shown in Table 2, has also been awarded GRAS status by FDA and can also be used in the invention.

The three (3) D-βOHB moieties are the horizontally arranged 4-carbon structures on the right. They are linked to the glycerol moiety which is the three (3)-carbon structure arranged vertically at the far left of the structural formula. During digestion in the small intestine, the glycerol moiety is split away from the parent compound, and the three D-βOHB molecules are also split away and absorbed through the gut wall into the portal vein which transports them to the inferior vena cava from which they enter the systemic circulation. Here is another example of the necessity of esterifying D-βOHB (as a ketone ester) so that, after it has been ingested in deacidified form, it will be well tolerated by the gut.

While these sample esters are discussed in detail they are merely representative of the carrier vehicles that are used in the invention.

Water solubility The constituents of the two ketone esters discussed above, as shown by their structures in Tables 1 and 2,would be expected to be water-soluble.

The ketone ester (R)-3-hydroybutyl (R)-3-hydroxybutyrate, although somewhat water soluble, is not materially digested by stomach acid and yields the rises in ketone bodies in the blood shown below following oral ingestion and digestion in the small intestine.

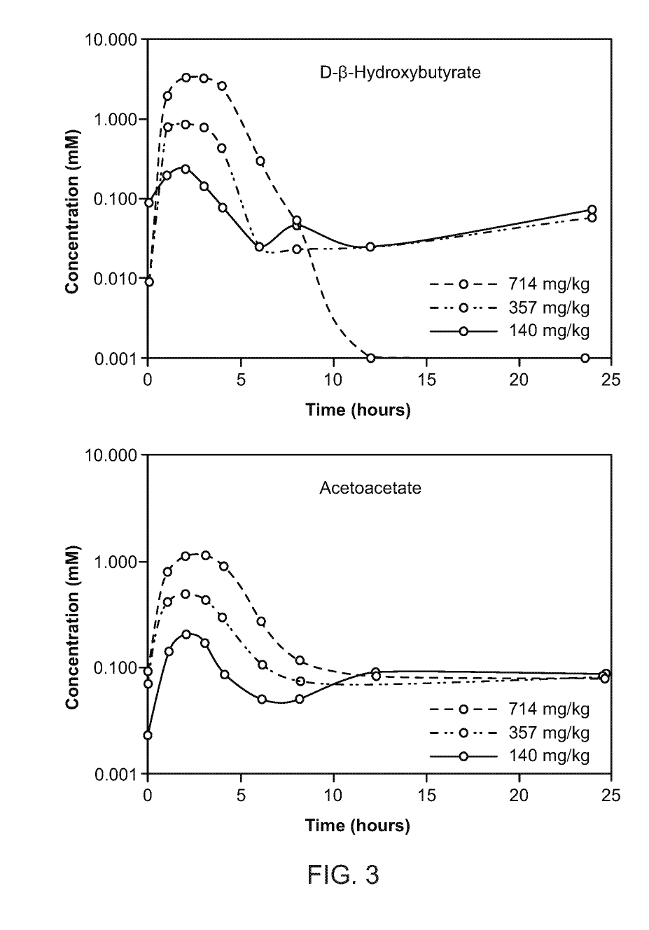

The graphs illustrated in FIG. 3 are from a paper on ketone monoester published by Kieran Clarke PhD, a professor at the University of Oxford and Vanitallie (Applicant herein). The graphs show that as the amount of ketone monoester (KME) taken by mouth (in mg/kg body weight) increases in quantity the rise in the blood level of the two ketone bodies (D-βOHB and AcAc) increases proportionately. The graphs show how high the levels of each of these two ketone bodies get and how long the elevations in blood levels of the ketone bodies last. The graphs are on a logarithmic scale—a legitimate way of expressing the findings.

The nutritional agents of the invention can be administered as a mixed-in component of a palatable liquid drink or a solid food product.

There are a number of ways in which the ketone ester is delivered orally. The basic principles followed is to combine the carrier vehicle; namely, the ketone ester which consists of the acidic ketone body combined with a neutralizing molecule like glycerol, in a liquid or solid medium that improves the taste and consistency and is gastrointestinally tolerable and comfortable. A variety of flavors are used to make the agent more palatable to the user.

After the invention nutritional agent is ingested by the user the carrier vehicle is digested and metabolized to release said ketone body for absorption from the intestinal lumen into the portal vein for rapid and efficient transfer into the circulatory system.

It is known that shortly after a ketone ester has been orally ingested, there is a substantial and rapid rise in the ketone (D-βOHB) concentration in the circulating bloodstream. The pattern of the rise confirms the rapid and efficient transfer of the ketone body into the blood circulation is shown in FIG. 3.

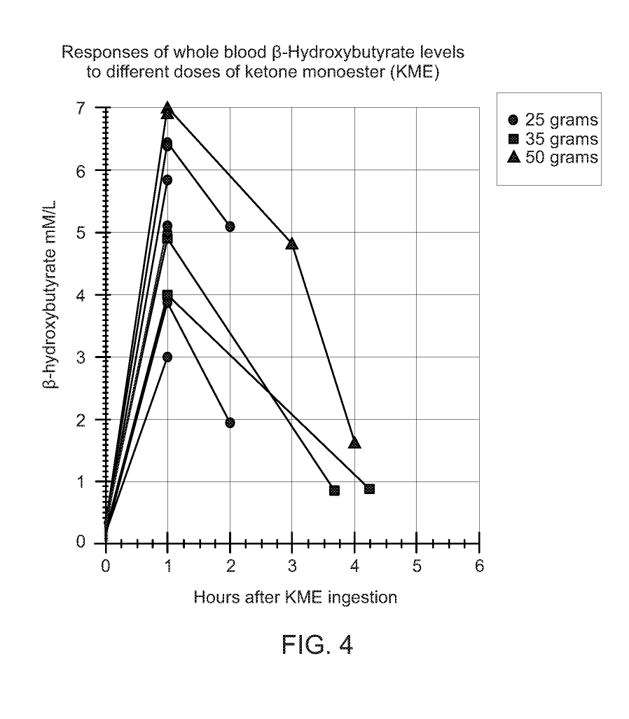

FIG. 4 is a graphic illustration of the time course of changes in blood D-βOHB concentration after a patient ingested increasing quantities of D-βOHB [ketone] monoester [KME] on successive days. Note that the peak levels achieved seem to occur about 1 hour after KME ingestion. During the three hours after ingestion of the largest quantity (50 g), D-βOHB blood concentrations remained between 4.8 and 7 mM [taken from the 2014 paper by Newport, MT et al. A new way to produce hyperketonemia: Use of ketone ester in a case of Alzheimer's disease. Alzheimer's & Dementia 2014;1-5].

Because of the water-solubility of D-βOHB and AcAc, both ketone bodies would necessarily enter the portal venous system after leaving the lumen of the small intestine. They would not travel in the lymphatic system, which is designed to transport the relatively insoluble lipids to the thoracic duct and thence into the general circulation in the form of microscopic lipid particles known as chylomicrons. It is because of their solubility and molecular size and configuration that the ketone bodies, D-βOHB and AcAc, are able to cross the intact blood-brain barrier (BBB) and serve as an essential alternate fuel to help meet the brain's high and constant energy needs—particularly on occasions when glucose is in short supply, as occurs (for example) during prolonged fasting. Fatty acids and fats generally are unable to cross the BBB.

### Early and Late Phase Reactions

The immune system's response to allergen exposure can be divided into two phases. The first is immediate hypersensitivity or the early phase reaction, which occurs within 15 minutes of exposure to the allergen. The second, or late phase reaction, occurs 4-6 hours after the disappearance of the first phase symptoms and can last for days or even weeks. During the early phase reaction chemical mediators released by mast cells including histamine, prostaglandins, leukotrienes and thromboxane produce local tissue responses characteristic of an allergic reaction. In the respiratory tract for example, these include sneezing, edema and mucus secretion, with vasodilatation in the nose, leading to nasal blockage, and bronchoconstriction in the lung, leading to wheezing. During the late phase reaction in the lung, cellular infiltration, fibrin deposition and tissue destruction resulting from the sustained allergic response lead to increased bronchial reactivity, edema and further inflammatory cell recruitment. These observations suggest that IgE is instrumental in the immune system's response to allergens by virtue of its ability to trigger mast cell mediator release, leading directly to both the early and late phase reactions. Similar phase reactions presumably involve basophils and eosinophils.

```
TABLE 3
Allergen --->
  IgE-antibody --->
    Mast cell/Basophil/Eosinophil -/--> (Here (“/”) is where ketone
    bodies seem to act, preventing or reducing the release of symptom-
    producing mediators.)
      Mediators [symbol]
        Inflammation --->
          Symptoms and Signs of Disease
```

In Table 3 above, the effect of ketone-body treatment is at the point where the Mast cell/Basophil/Eosinophil cell—→Mediators. Ketone treatment according to the invention inhibits/block this release thereby preventing inflammation and symptoms of the disease.

The ketone body acts to stabilize and render unresponsive to activation of mast cells, basophils, and eosinophils which, upon exposure to one or more allergens(antigens) react by extruding their granules. The effect of ketone bodies is to block or in varying degrees, inhibit such granular extrusion. Consisting of histamine and other bioactive substances, these granules act on the body's cells and organs either locally or systemically to produce characteristic sings and symptoms that physicians recognize as being manifestations of sensitivity/allergy.

Target blood levels of the ketones used in the invention to stabilize mast cells, is between 0.5 mM to 7 mM. Administration orally of a ketone ester that generates ketone body levels (as D-βOHB) of 5±2 mM would be preferred to a ketogenic diet or a fasting regiment. Raising the ketone body level above 7 mM might produce some undesirable ketoacidosis. Less that 3 mM might not be high enough to obtain the desired effect.

The classic ketogenic diet, first used to prevent epileptic seizures in the 1920's, provided about 90% of its calories as fat, about 8% as protein, and 2% as carbohydrate. If a significant portion of the fat was in the form of long-chain saturated fatty acids, this would cause low-density lipoprotein (LDL) cholesterol to rise, increasing the risk of atherogenesis and cardiovascular disease (coronary atherosclerosis and cerebrovascular atherosclerosis). The high-fat, saturated fat-rich diet would also promote vascular inflammation and increase risk of development of neurodegenerative disease in the elderly. The classic ketogenic diet is difficult to prepare properly and adhere to. Other adverse side effects may occur, such as nephrolithiasis—which appears to be associated with chronic dehydration.

Use of such a diet to prevent or treat allergic disorders and certain forms of fibrosis would not be practicable or desirable, particularly when high plasma ketone body levels can be readily achieved by consumption of suitable portions of ketone ester every 3 to 4 hours without the necessity of changing the remaining diet. Early research suggests that the two ketone esters studied to date are well tolerated by the human gastrointestinal tract.

The foregoing description of various and preferred embodiments of the present invention has been provided for purposes of illustration only, and it is understood that numerous modifications, variations and alterations may be made without departing from the scope and spirit of the invention as set forth in the following claims.

What is claimed is:

1. A nutritional agent that orally delivers a therapeutically effective amount of a ketone body in a carrier vehicle to prevent and treat hypersensitivity and allergic disorders.

2. The nutritional agent according to claim 1, wherein said ketone body is selected from the group consisting of D-β-hydroxybutyrate (D-βOHB), acetoacetate (AcAc), acetone and any of their isoforms.

3. The nutritional agent according to claim 1 wherein said disorders include hay fever, asthma, allergic eczema, atopic dermatitis, medication allergy and systemic anaphylaxis.

4. The nutritional agent according to claim 1, wherein said carrier vehicle is a gastro-intestinally benign form.

5. The nutritional agent according to Clam 4, wherein said carrier vehicle is a ketone ester or a ketone salt, safe for human consumption.

6. The nutritional agent according to claim 5, wherein said ketone ester is selected from the group consisting of 1, 3 butanediol monoester of D-βOHB and glycerol tri-ester of betahydroxbutyrate.

7. The nutritional agent according to claim 5, wherein said ketone salts are selected from the group consisting for the ketone body salts of Na⁺, K⁺, Ca²⁺, Mg²⁺.

8. The nutritional agent according to claim 1, wherein said carrier vehicle further includes at least one of the components selected from the group consisting of glycerol, phospholipids, triglycerides, partial glycerides, lipoproteins, medium-chain fatty acids and lauric acid.

9. The nutritional agent according to claim 1, wherein said carrier vehicle is digested and metabolized to release said ketone body for intestinal absorption and transfer into the circulatory system.

10. The nutritional agent according to claim 1, further comprising a liquid beverage.

11. The nutritional agent according to Clam 1, further comprising a food product.

12. The nutritional agent according to claim 1, wherein said ketone body acts to stabilize and render unresponsive the activation of mast cells, basophils and eosinophils, which upon exposure to one or more allergens/antigens respond by extruding their granules.

13. The nutritional agent according to claim 12, wherein the effect of said ketone body is to block or inhibit granular extrusion.

14. The nutritional agent according to claim 12, wherein said ketone body further inhibits said mast cell/basophil/eosinophil and any other cells degranulation and release of histamine, prostaglandins and other bioactive agents that are secreted in response to allergens and allergen-like agents.

15. The nutritional agent according to Clam 1, wherein said therapeutically effective amount of said ketone body provides blood levels in the range of 0.5 mM to 7 mM.

16. A method to prevent and treat allergy hypersensitivity disorders comprising the steps of:
administering a nutritional agent that orally delivers a therapeutically effective amount of a ketone body in a carrier vehicle;
wherein said carrier vehicle is digested and metabolized to release said ketone body for intestinal absorption and efficient transfer into the circulatory system;
wherein said ketone body acts to stabilize mast cells, basophils and eosinophils, and to further inhibit the release of bioactive agents from said mast cells, basophils and eosinophils.

17. The method according to claim 16, wherein said nutritional agent provides blood levels of said ketone body in the range of 0.5 mM to 7 mM.

18. The method according to claim 16, wherein said nutritional agent is taken by a user every 3 to 4 hours to keep blood levels of said ketone body in the range of 0.5 mM to 7 mM.

19. The method according to claim 16, wherein said ketone body is released for absorption from the intestinal lumen into the portal vein for rapid transfer into the circulatory system.

20. The method according to claim 16, wherein said bioactive agent is selected from the group consisting of histamine, prostaglandins and other bioactive agents that are secreted in response to allergens and allergen-like agents.
